(19) United States
(12) Patent Application Publication
(10) Pub. No.: US 2026/0272504 A1
(43) Pub. Date: Sep. 17, 2026

(54) AORTIC SEPTAL FENESTRATION DEVICE

(71) Applicant: The Regents of The University of Michigan, Ann Arbor, MI (US)

(72) Inventors: Shinichi FUKUHARA, Ann Arbor, MI (US); Gardner YOST, Ann Arbor, MI (US); Jeffrey Stephen PLOTT, Algonac, MI (US)

(73) Assignee: The Regents of The University of Michigan, Ann Arbor, MI (US)

(21) Appl. No.: 19/471,606

(22) PCT Filed: Apr. 4, 2024
(86) PCT No.: PCT/US2024/023025

§ 371 Date: Oct. 1, 2025

Related U.S. Application Data
(60) Provisional application No. 63/457,212, filed on Apr. 5, 2023.

(51) Int. Cl. A61B 17/34; A61B 17/00

(52) U.S. Cl. CPC A61B 17/34; A61B 17/00234; A61B 2017/00323; A61B 2017/00566; A61B 2017/00778

(57) ABSTRACT

An aortic fenestration device having an outer sheath; a suction head being operably coupled to a distal end of the outer sheath and having a suction aperture; a fenestration element disposed within the outer sheath and extendable through the suction head to a position beyond the suction aperture to fenestrate tissue; a guidewire slidably disposed relative to the fenestration element and extendable to a position beyond the suction aperture and a distal end of the fenestration element; and a suction source operably coupled to the outer sheath and configured to exert suction pressure at the suction aperture of the suction head to selectively couple the suction head to the tissue via suction pressure as the tissue is fenestrated. The fenestration element can comprise a needle-wire and needle-wire catheter or a hollow needle.

### CROSS-REFERENCE TO RELATED APPLICATIONS

This application claims the benefit of U.S. Provisional Application No. 63/457,212, filed on Apr. 5, 2023. The entire disclosure of the above application is incorporated herein by reference.

### FIELD

The present disclosure relates to aortic dissection treatment and, more particularly, relates to an aortic septal fenestration device for treatment of aortic dissections and/or associated complications.

### BACKGROUND AND SUMMARY

This section provides background information related to the present disclosure which is not necessarily prior art. This section provides a general summary of the disclosure and is not a comprehensive disclosure of its full scope of all its features.

Aortic dissection occurs when there is separation between the layers of the aortic wall, as illustrated in FIG. 1A. There are three aortic layers: intima (innermost layer, one side faces the lumen of the vessel), media (middle layer), and adventitia (outermost layer). A tear in the intima leads to aortic dissection as blood fills the space between the layers of aorta (creating a false lumen in the process). Pressurized blood within this false lumen can promote progression of the dissection.

Aortic dissections can prevent proper blood flow, which can lead to decreased blood flow to organs—called malperfusion—increasing the risk for severe organ damage due to ischemia. Dissections can compress the true lumen of the aorta which can significantly impair the flow of blood at branch vessels (vessels taking blood from the aorta to the organs and extremities). Blood flow to branch vessels may be impaired by invagination of the false lumen into the true lumen of the branch vessel.

The outer wall of the false lumen (media and adventitia) is weaker than the three layers together, generating risk of rupture or aneurysmal degeneration for patients with aortic dissection. Additionally, a pressurized false lumen is at increased risk for other serious and life-threatening complications like malperfusion or aortic rupture.

Aortic dissections are classified as one of two types. Namely, as illustrated in FIG. 1B, type A aortic dissections involve the aorta proximal to the brachiocephalic artery (closer to the heart), which require immediate surgery. As illustrated in FIG. 1C, type B aortic dissections involve the descending aorta and are distal to the left subclavian artery. Malperfusion can occur as a result of both types of aortic dissections. If malperfusion is not present, type B dissections may be treated without surgical intervention. Furthermore, type B aortic dissection can be subclassified as acute (less than 3 months from the onset) and chronic type B aortic dissection (3 months or greater from the onset) based on the chronicity of the aortic dissection. While one of main reasons requiring surgical intervention for “acute” aortic dissection is malperfusion, the most common reason for surgical intervention in patients with “chronic” type B aortic dissection is aortic aneurysm formation due to the loss of aortic wall integrity caused by the acute aortic dissection.

While some acute type B aortic dissections can be managed with medical management, other cases of acute type B dissections with complications such as malperfusion, aortic rupture, refractory hypertension and relentless pain and all acute type A dissections require surgical intervention. The mainstay of surgical intervention for acute type A dissections is open heart surgery. Surgical intervention for acute type B dissection with complications involves the endovascular introduction of a stent placed across the dissection to cover the entry tear creating the dissection and allow blood to only flow through the true lumen. However, stent therapy requires introduction of a hardware in the friable dissected aorta, not necessarily effective for correction of malperfusion and time-consuming. Additionally, stent therapy alone has risk of complications such as stent-induced new entry tear as a result of introducing a hardware in a friable aorta and lifelong risk of device infection. This new entry tear results in additional malperfusion risk and future aneurysm formation. Patients with stent therapy requires lifelong prophylactic antibiotics every time receiving procedures for stent device infection prevention such as dental procedures. More effective and ideal therapeutic option for correction of malperfusion is aortic fenestration, which is the generation of an additional opening in the aortic septum to restore blood flow to the true lumen and end-organs. However, aortic fenestration is a very high-risk and technically challenging procedure requiring off-label device usage. There have been no dedicated medical devices for this purpose.

Operative treatment for chronic type B aortic dissection-related aortic aneurysm is associated with extremely high mortalities and complications rates. Therefore, a less invasive therapy such as endovascular therapy using a stent would be ideal in high-risk patients with comorbidities. However, stent therapy is often not effective for chronic type B aortic dissection because of the presence of stiff aortic septum, which impedes proper stent apposition against the aortic wall. Unfortunately, a significant number of patients are considered unsuitable for operative or less invasive stent treatment due to comorbidities and lack of suitable anatomic sites for stent deployment. In cases where patients are unsuitable for standard open-heart surgery or stent therapy, a procedure can be done to provide better stent attachment sites. Aortic fenestration is also very effective in this clinical setting with chronic type B aortic dissection by optimizing the seating of the endograft.

Aortic fenestration is performed as an endovascular procedure. It is currently a highly technically demanding procedure without dedicated devices available which many physicians do not feel comfortable performing in the setting of aortic dissection. Therefore, it is presently not performed at the majority of institutions. Because of this, malperfusion from “acute” aortic dissection may never be fully treated resulting in extremely high mortality, and stent-graft placement for “chronic” type B aortic dissection may not be optimized or is not even offered.

Current techniques to perform aortic fenestration involve using a catheter with energy source (electrocautery, radiofrequency ablation, laser) to cross the septum, or using an endovascular needle to puncture the septum. None of these devices is meant for aortic fenestration and these are off-label device usage procedure. Furthermore, these techniques are universally challenging and not widespread. In chronic septa, tissue between the true and false lumen is thick, making fenestration more difficult. A needle or catheter with energy source may be difficult to control, and apposition against the septum may be technically challenging. This can lead to possible life-threatening complications like a serious injury to the aorta that could lead to aortic rupture.

In acute aortic dissections, the septum is often mobile, the true lumen may be extremely collapsed and the false lumen compressible. This means that, when using an off-label septal fenestration technique such as a simple needle, capturing the target aortic septum can be challenging and there is increased risk of puncturing the aortic side wall, which can cause significant hemorrhage.

According to the principles of the present teachings, an aortic fenestration device is provided having advantageous construction and methods of use. In some embodiments, the aortic fenestration device comprises an outer sheath, a suction head being operably coupled to a distal end of the outer sheath and having a suction aperture, a fenestration element disposed within the outer sheath and extendable through the suction head to a position beyond the suction aperture to fenestrate tissue, a guidewire slidably disposed relative to the fenestration element and extendable to a position beyond the suction aperture and a distal end of the fenestration element, and a suction source operably coupled to the outer sheath and configured to exert suction pressure at the suction aperture of the suction head to selectively couple the suction head to the tissue via suction pressure as the tissue is fenestrated. The fenestration element can comprise a needle-wire and needle-wire catheter or a hollow needle.

In some embodiments, the aortic fenestration device comprises a sheath containing at least one lumen; a suction head being operably coupled to a distal end of the sheath, the suction head having an internal lumen and a suction aperture; a needle-wire catheter slidably disposed within the sheath and extendable through the suction head to a position beyond the suction aperture; a needle-wire slidably disposed within the needle-wire catheter and extendable through the needle-wire catheter to a position beyond the suction aperture and a distal end of the needle-wire catheter; and a suction source operably coupled to the sheath to exert suction pressure at the suction aperture of the suction head to selectively couple the suction head to tissue via suction pressure.

In another embodiment, the aortic fenestration device comprises a sheath containing at least one lumen; a suction head being operably coupled to a distal end of the sheath, the suction head having an internal lumen and a suction aperture; a hollow needle slidably disposed within the sheath and extendable through the suction head to a position beyond the suction aperture; a hollow needle advancement slider and external handle to control the orientation and positioning of the hollow needle; a guidewire slidably disposed within the hollow needle and extendable through the hollow needle to a position beyond the suction aperture and a distal end of the hollow needle; and a suction source operably coupled to the sheath to exert suction pressure at the suction aperture of the suction head to selectively couple the suction head to tissue via suction pressure.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C illustrate aortic dissections, including type A (FIG. 1B) and type B (FIG. 1C).

FIG. 2 illustrates a perspective view of an aortic fenestration device according to some embodiments of the present teachings.

FIG. 3 illustrates a plan view of the aortic fenestration device, with portions removed, according to some embodiments of the present teachings.

FIG. 4 illustrates a valve port device.

FIG. 5 illustrates an enlarged view of a suction head according to some embodiments of the present teachings.

FIG. 6 illustrates an enlarged view of the suction head according to some embodiments of the present teachings.

FIG. 7 illustrates an enlarged side cross-sectional view of the suction head according to some embodiments of the present teachings.

FIG. 8 illustrates an enlarged end cross-sectional view of the suction head according to some embodiments of the present teachings.

FIG. 9 illustrates an enlarged perspective view, with portions in hidden view, of the suction head according to some embodiments of the present teachings.

FIG. 10 illustrates a perspective view of the aortic fenestration device according to some embodiments of the present teachings.

FIGS. 11-17 illustrate a series of operational views of the aortic fenestration device according to some embodiments of the present teachings.

FIG. 18A illustrates a perspective view of an aortic fenestration device having an articulating section according to some embodiments of the present teachings.

FIG. 18B illustrates an enlarged view of the articulating section according to some embodiments of the present teachings.

FIG. 19 illustrates a plan view of the aortic fenestration device having the articulating section according to some embodiments of the present teachings.

FIG. 20 illustrates an enlarged side cross-sectional view of the suction head according to some embodiments of the present teachings.

FIG. 21 illustrates a side cross-sectional view of the handle assembly according to some embodiments of the present teachings.

FIGS. 22A-22B illustrate a series of operational views of the aortic fenestration device showing extension of the needle and guidewire, respectively, according to some embodiments of the present teachings.

FIGS. 23A-23B illustrate enlarged views of the guide features and suction channels according to some embodiments of the present teachings.

FIG. 24 illustrates an enlarged cross-sectional view of the suctional channel according to some embodiments of the present teachings.

FIG. 25 illustrates an enlarged perspective view of the suctional channel according to some embodiments of the present teachings.

FIG. 26 illustrates an enlarged end cross-sectional view of the suction head according to some embodiments of the present teachings.

FIGS. 27A-27D illustrate a series of operational views of the aortic fenestration device and needle according to some embodiments of the present teachings.

FIG. 28 illustrates an enlarged perspective view of the suction head having bipolar electrodes according to some embodiments of the present teachings.

FIG. 29 illustrates an enlarged perspective view of the suction head having a balloon inflation mechanism according to some embodiments of the present teachings.

FIG. 30A illustrates an enlarged perspective view of the suction head having a laser according to some embodiments of the present teachings.

FIG. 30B illustrates a perspective view of an aortic fenestration device having a laser according to some embodiments of the present teachings.

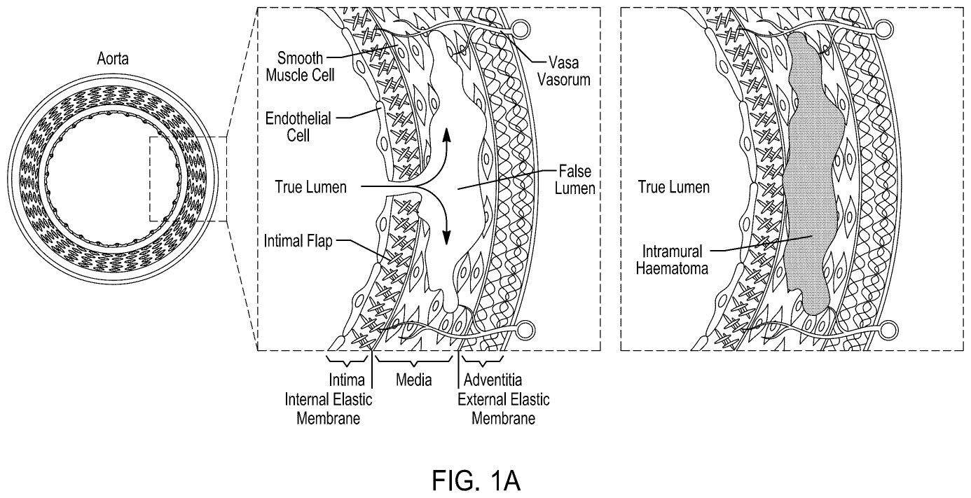

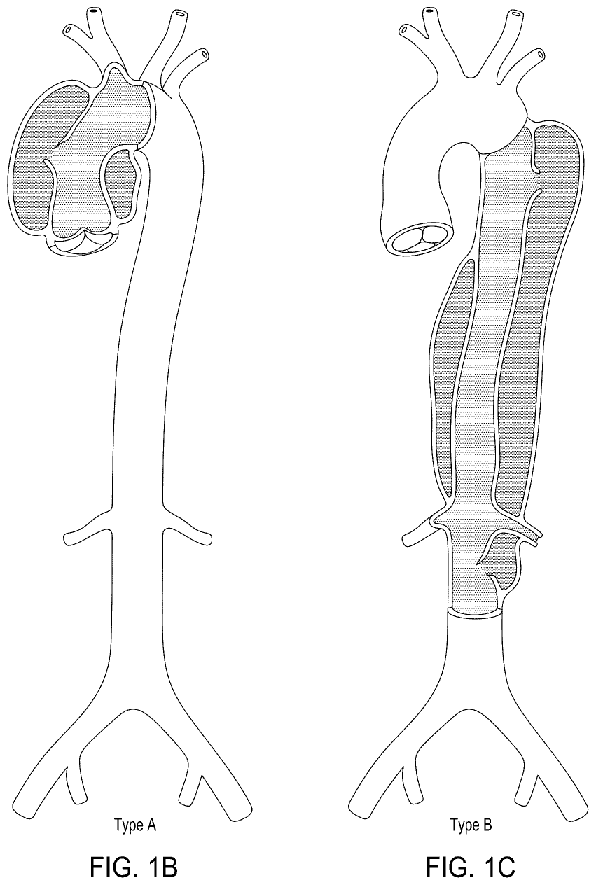

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

According to the principles of the present teachings, an endovascular catheter device and method of using the same are provided to decrease the technical demand of aortic fenestration in order to make it a more accessible procedure. The endovascular, catheter-based device is configured to generally be advanced into the aorta, vessel, or other tubular anatomic structure until it reaches the area where the fenestration will be performed. In some embodiments, suction is applied to attach the device head to the aortic septum; the suction is delivered to the device head by a small-bore catheter and is externally generated. In some embodiments, a needle-wire is then advanced through the catheter and pierces the aortic septum. In some embodiments, a needle-catheter is slid over the needle-wire into the false lumen. The needle-wire is removed, and a guidewire is placed through the needle-catheter into the false lumen. The device is removed over the guidewire, leaving the guidewire in place in the false lumen.

In some embodiments, a cannula, or hollow needle, is used to pierce the aortic septum instead of the needle-wire and a guidewire is advanced through the inner lumen of the hollow needle into the false lumen. The device is then removed over the guidewire, leaving the guidewire in place in the false lumen. While other devices do exist for aortic fenestration, none of these devices are capable of generating suction, or provide the benefits and advantages of the present teachings.

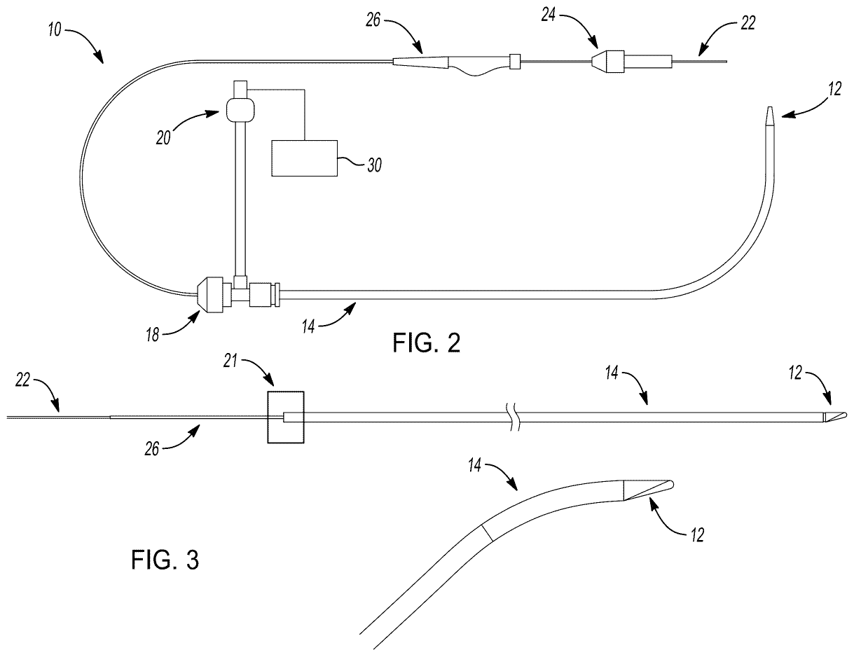

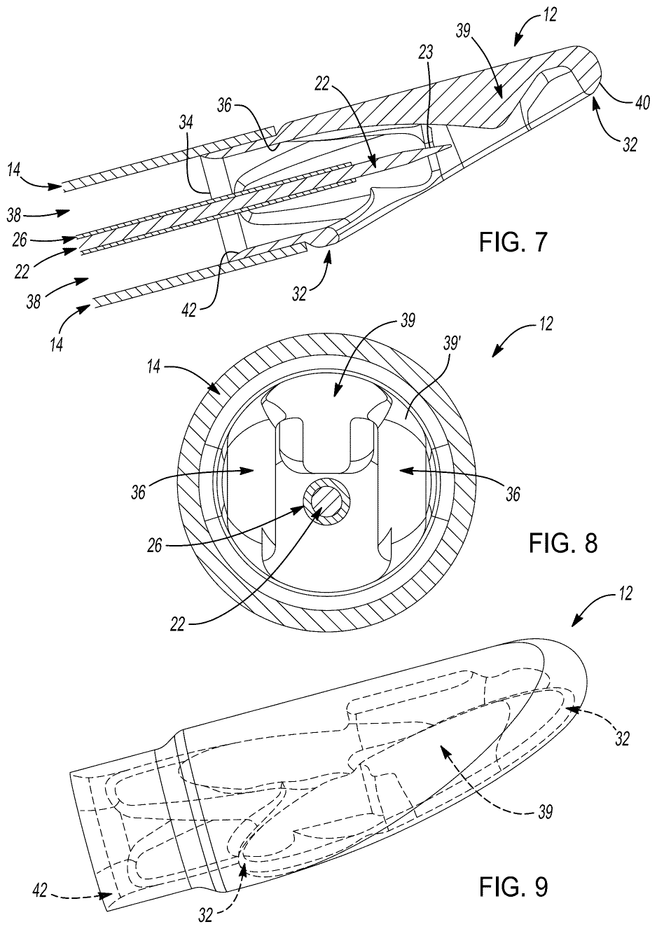

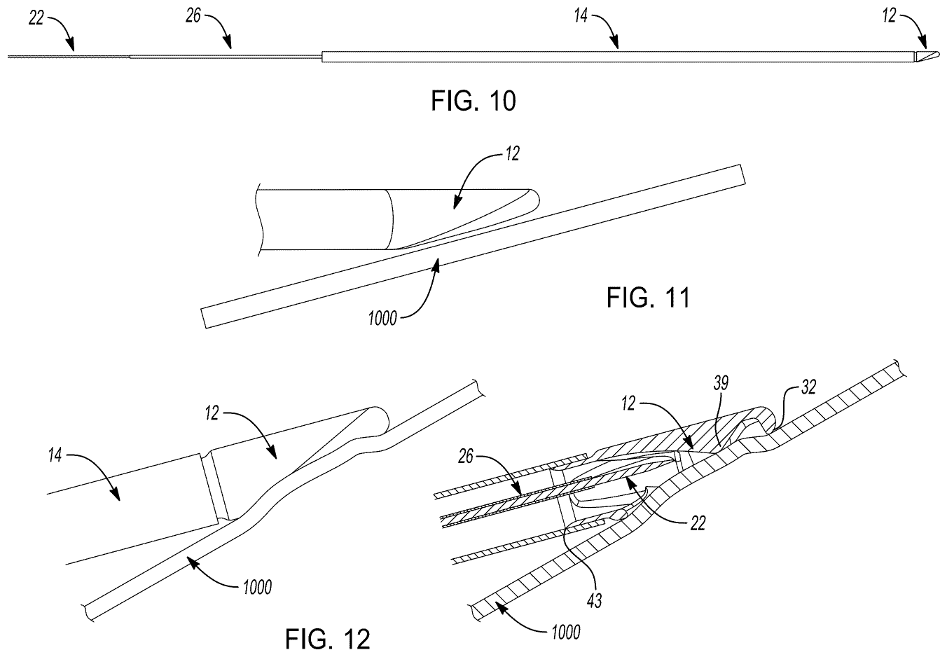

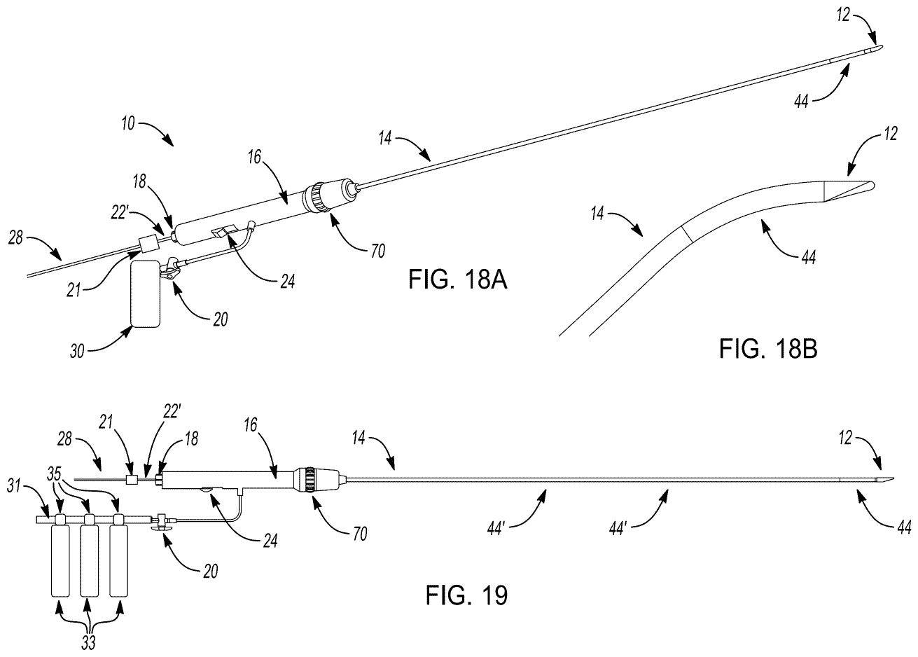

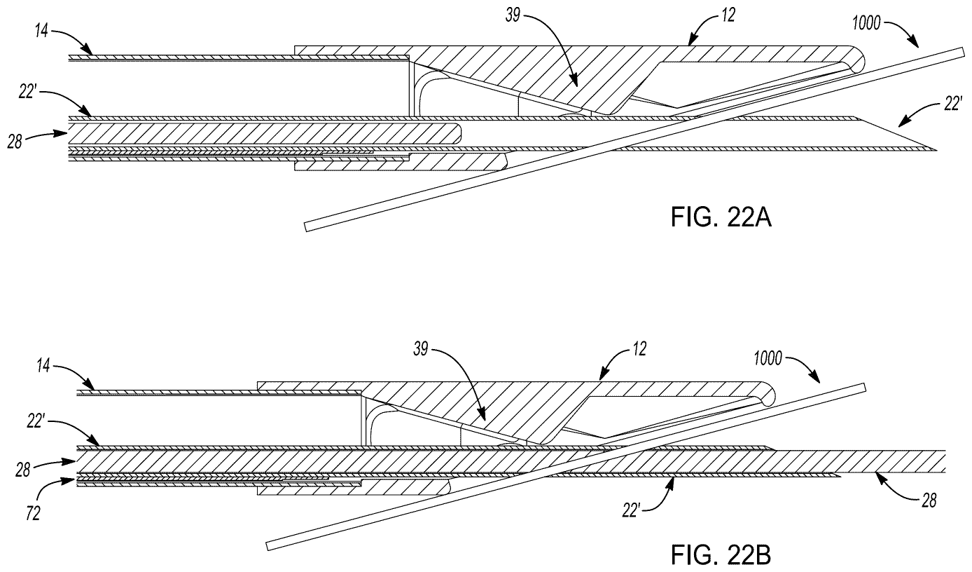

More particularly, according to the principles of the present teachings, an endovascular, catheter-based device (herein after referred to as the aortic fenestration device 10) configured, adapted, and operable to create fenestration in aortic septum 1000 is provided. In some embodiments, generally, the aortic fenestration device 10 includes components which permit temporary and/or selective attachment to the aortic septum 1000 by suction. In some embodiments, alternative means of temporary and/or selective attachment of aortic fenestration device 10 to the aortic septum 1000 can be used, such as but not limited to balloon attachment (see FIG. 29) and other systems. While the distal end of the aortic fenestration device 10 is attached to the aortic septum 1000 via a suction head in some embodiments, a fenestration element, such as a needle-wire (FIGS. 2-17), hollow needle (FIGS. 18A-27D), or laser fenestration component (see FIG. 30A-30B), is advanced to provide access to the false lumen. Once the false lumen is accessed, a guidewire is advanced through the aortic fenestration device 10 into the false lumen and the aortic fenestration device 10 is removed.

In some embodiments, the aortic fenestration device 10 provides access to the false lumen. In this case, “access” refers to the placement of a guidewire in the false lumen. In some cases, the aortic fenestration device 10 will be advanced into the false lumen from the access vessel and will be used to cross the septum into the true lumen. Guidewire access permits the surgeon to proceed to perform a range of additional therapeutic and/or lifesaving procedures, such as but not limited to false lumen decompression, dilation of fenestration, stenting of branch vessel, septostomy, and diagnostic angiography.

Although there are currently products that provide the ability to fenestrate an aortic septum 1000 with a needle or with a laser, there are no products which provide a stable and reliable apposition to the septum in a controlled manner either by generating suction or other means, such as balloon inflation. In some embodiments of the present teachings, the aortic fenestration device 10 uses one or more apertures on the suction head to attach to the aortic septum 1000. The needle or laser is then advanced through or adjacent to the suction head to create septal fenestration. In some embodiments, the suction is delivered to the suction head via small bore catheter and is externally generated. In some embodiments, both the suction and the needle or laser fenestrator are delivered via a catheter or multiple separate catheters. These catheters can also be used to advance the aortic fenestration device 10.

With particular reference to FIGS. 2 and 3, in some embodiments, the aortic fenestration device 10 can comprise a device or suction head 12, outer sheath or housing 14, a needle exit locking mechanism 18, a Luer suction port (e.g., Hemostatic valve) 20, needle-wire 22 having a proximal end with advancement mechanism device 24, needle-wire catheter 26, a guidewire 28, and locking suction syringe or other means of generating suction 30.

With particular reference to FIGS. 18A-22B, in some embodiments, the aortic fenestration device 10 can comprise suction head 12, outer sheath 14, a handle 16, needle exit locking mechanism 18, Luer suction port (e.g., Hemostatic valve) 20, needle 22′ having a proximal end with advancement mechanism 24, a guidewire 28, and locking suction syringe or other means of generating suction 30.

### Suction Head

In some embodiments, as illustrated in FIGS. 2-3, 5-10, 18A-20, and 22A-26, suction head 12 can comprise one or more suction apertures 32 to facilitate a selective, temporary, and/or reliable suction connection or coupling of suction head 12 with aortic tissue 1000. The size of suction aperture 32 is maximized to improve suction transmission and attachment of suction head 12 to aortic septum 1000. In some embodiments, the shape of suction aperture 32 is circular, ellipsoid, oval, or rounded to enhance tissue engagement and/or accommodate tissue irregularities during suction connection or coupling. In some embodiments, suction aperture 32 is ovoid and smooth walled in shape to prevent loss of suction at corners and angles. In some embodiments, the inner walls of the suction aperture 32 are serrated, textured, and/or grooved to improve attachment and fixation to septum 1000 under negative pressure.

In some embodiments, suction head 12 can further comprise a needle catheter aperture 34. In some embodiments, suction head 12 can comprise an atraumatic/blunted shaped head to permit improved ease of movement. In some embodiments, suction head 12 can plug into or otherwise be coupled to other catheter portions.

During operation, suction head 12 delivers suction from catheters to the aortic septum 1000 and may remain in place while needle-wire 22, needle 22′, and/or needle-wire catheter 26 are advanced across aortic septum 1000.

### Outer Sheath

In some embodiments, outer sheath 14 can comprise an outer sheath and/or housing configuration that spans the distance between a user interface (e.g., handle 16) and suction head 12. In some embodiments, outer sheath 14 can comprise a single lumen 38 catheter for passage of needle-wire catheter 26 and/or needle 22′ as well as negative pressure. In some embodiments, outer sheath 14 can comprise a second lumen to be utilized for communication of negative pressure between suction source 30 and suction head 12.

In some embodiments, outer sheath 14 can comprise a semi-rigid outer wall 40 capable of resisting crush while transmitting negative pressure, being deflectable and/or steerable, and while being within pressurized aorta. In some embodiments, outer sheath 14 is composed of one or more layers to achieve the desired mechanical properties. In some embodiments, outer sheath 14 may comprise one or more materials with different hardnesses at different locations, such as but not limited to medical grade polyether block amide or nylon. In some embodiments, one or more sections may have material hardnesses between shore D 20-90, although other hardnesses may be used. In some embodiments, outer sheath 14 may be made from a medical grade thermoplastic material and may include an integral metal braid or coil structure to help provide adequate strength while maintaining flexibility. In some embodiments, a portion of outer sheath 14 may include an indicator component and/or radiopaque markers showing directionality of distal pre-curvature or bend and/or permit detection of real-time location and/or positioning of suction head 12 or other notable portion of aortic fenestration device 10.

In some embodiments, outer sheath 14 can be steerable and/or can include pre-curvature. In some embodiments, the shape and steerability of outer sheath 14 enable apposition of the suction head 12 to the aortic septum 1000. In some embodiments, as illustrated in FIGS. 18A-19, aortic fenestration device 10 is configured to articulate or deflect a portion 44, such as a distal portion and/or discrete or continuous intermediate portions 44′, of the outer sheath 14. That is, the articulation may be along one or more sections of outer sheath 14 depending on the navigational requirements of the surgical application. The magnitude and direction of articulation/deflection of outer sheath 14 may be controlled by an outer sheath articulating assembly 70 (FIGS. 18A, 19, and 21) such that the user may select the degree of articulation/deflection by greater or lesser manipulation of outer sheath articulating assembly 70. In some embodiments, the articulation/deflection of outer sheath 14 can be controlled by, for example, utilizing one or more pull wires 72 (FIGS. 21 and 26) integrated within outer sheath 14. In some embodiments, as illustrated in FIG. 26, pull wire 72 may be located along the side of outer sheath 14 (i.e., offset from a longitudinal axis) or within the wall of outer sheath 14 on the same side as aperture opening 32, to exert a bending force to facilitate articulation towards the direction of suction aperture 32. Other orientations and quantities of pull wire 72 may also be used to control articulation.

Pull wires 72 can be connected to a mechanism of outer sheath articulating assembly 70 within handle 16. In some embodiments, outer sheath articulating assembly 70 can comprise a rotating member 74, such as a rotating member threadedly engaging a corresponding thread system 76 which moves fore and aft in response to rotating member 74, thereby driving pull wires 72 resulting in a bending force thereby articulating/deflecting the corresponding section or zone of outer sheath 14. However, it should be understood that alternative drive systems may be used, such as a thumb toggle. In some embodiments, outer sheath articulating assembly 70 can maintain the articulation of outer sheath 14 even when not actively held. This allows the operator to set the desired articulation of outer sheath 14, and then frees up their hand to perform other tasks.

### Suction Head Features

In some embodiments, externally generated suction is delivered to the suction head 12. In some embodiments, the suction head 12 is coupled to and continuous and/or coaxial with the outer sheath 14 and possesses an internal suction head lumen 36 continuous with the internal lumen 38 of the outer sheath 14 (e.g., outer sheath lumen 38). The internal suction head lumen 36 terminates at suction aperture 32, which is located on a single face of suction head 12 and which functions to transmit suction and route the needle-wire 22/needle 22′ and the needle-wire catheter 26. In some embodiments, one or more suction head lumens, channels, and/or open spaces 36 facilitate transmission of suction pressure within suction head 12 to suction aperture 32 to promote suction engagement with aortic septum 1000.

In some embodiments, as illustrated in FIGS. 8 and 23A-25, suction head 12 can comprise one or more suction lumens or ports 36 for transmitting suction pressure that is generated or supplied by suction source 30 and transmitted via fluidic channels within outer sheath 14 to suction head 12. More particularly, in some embodiments, suction is transmitted along outer sheath lumen 38 to one or more suction head lumens 36.

In some embodiments, the suction head 12 possesses one or more guide features 39. In some embodiments, guide feature 39 can comprise one or more protrusions and/or contoured surfaces in suction head 12 configured to direct the needle-wire 22, needle 22′, and/or needle-wire catheter 26 towards the center of the suction aperture 32 and/or suction head 12. Moreover, in some embodiments, guide feature 39 can comprise lateral features 39′ located laterally and proximally within suction head 12 configured to constrain the needle-wire 22, needle 22′, and/or needle-wire catheter 26 to the middle of suction head 12 and inhibit operational binding within suction head 12. In some embodiments, an additional feature can be located distally and centrally within suction head 12. Guide features 39, 39′ direct the needle-wire 22, needle 22′, and/or needle-wire catheter 26 downward (away from suction head 12) and/or centrally (along a longitudinal axis of suction head 12). This optimizes needle fenestration by 1) ensuring that needle-wire 22, needle 22′, and/or needle-wire catheter 26 exits the suction aperture 32 centrally, rather than at the tip or edge of the suction aperture 32 to prevent loss of suction by peeling the suction head 12 away from the aortic septum 1000 and 2) by making the angle at which the needle-wire 22/needle 22′ approaches the aortic septum 1000 less oblique, decreasing the force required to pass the needle-wire 22/needle 22′ through the septal tissue.

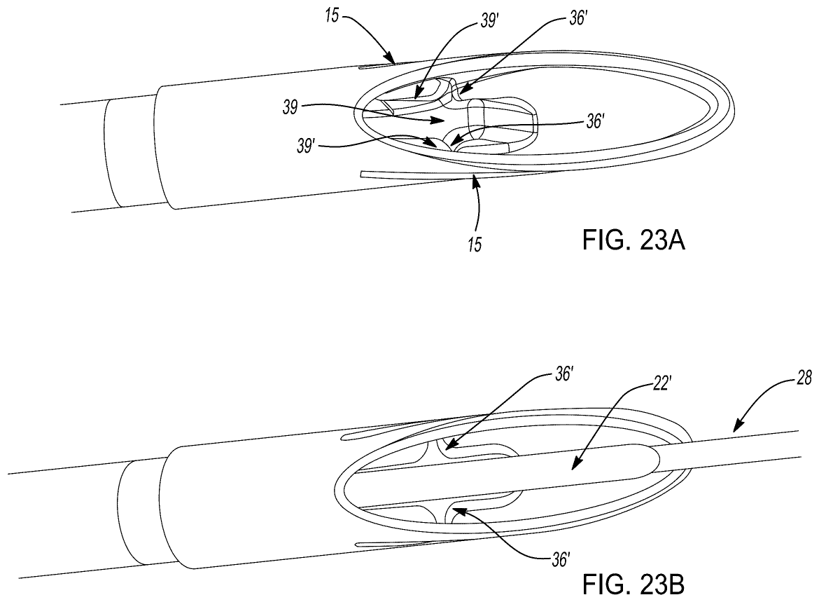

In some embodiments, as illustrated in FIGS. 23A-25, one or more suction head lumen 36 may be formed relative to guide features 39, 39′ ensuring that suction is delivered across the entire suction aperture 32 when tissue is engaged. For example, in some embodiments without adequate channels to permit suction passage, it may be possible for the tissue to seal off the most distal portion of the suction aperture 32 as it is pulled into the suction aperture 32. This could reduce the holding force between the septum 1000 and the suction aperture 32. Accordingly, in some embodiments as illustrated in FIGS. 23A-23B, suction head lumen 36′ can be formed along a space between central guide feature 39 and each lateral guide feature 39′, thereby ensuring suction passage therethrough.

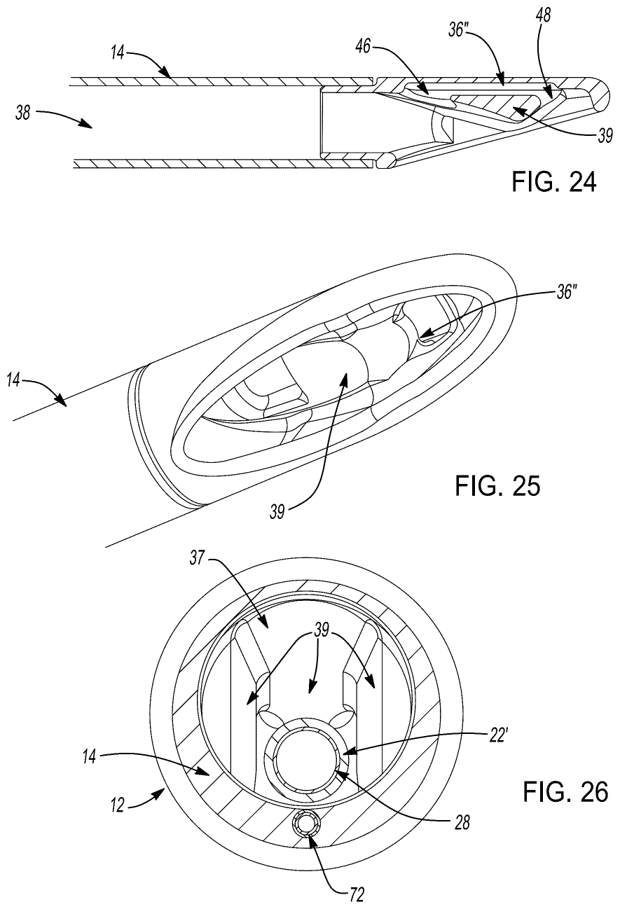

In some embodiments, as illustrated in FIGS. 24-25, suction head lumens 36 can be formed as one or more suction channels 36″ formed through a portion of guide feature 39 and/or suction head 12. In this way, even if flexible tissue is pulled into the orifice of the head, which may be obstructive to suction and could result in a reduction of holding force attaching the device to the tissue, the suction is able to pass from the proximal opening 46 of the suction channel 36″ to the distal opening 48, enabling suction to reach the distal end of the suction head 12.

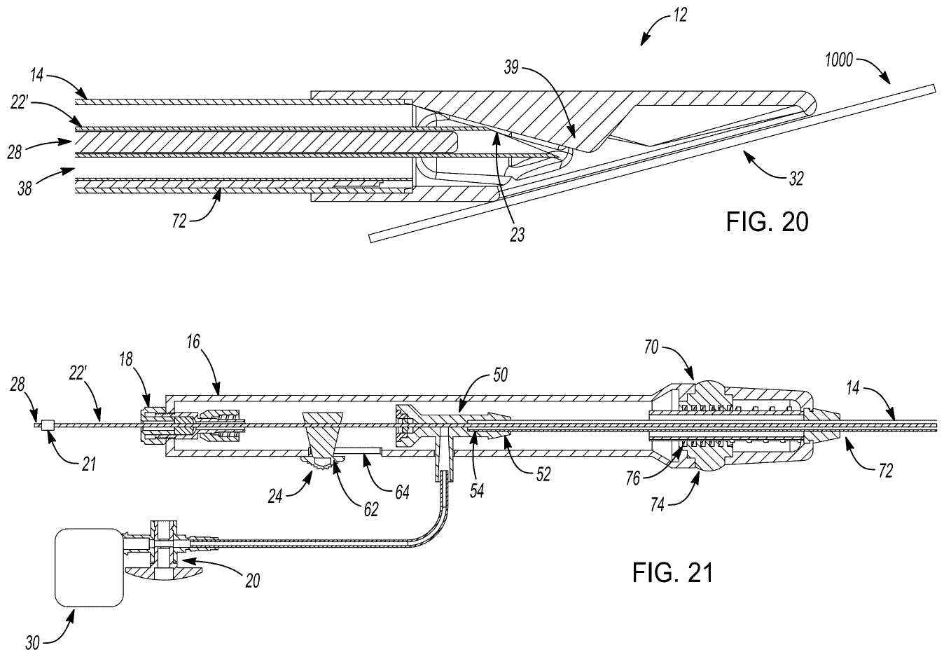

In some embodiments, as illustrated in FIG. 20, needle 22′ can comprise a beveled distal end or tip 23. In some embodiments, beveled distal end 23 can be beveled to define a sloped or sharpened tip generally formed at an inclined angle relative to guide feature 39 to promote smooth, generally non-binding, extension of needle 22′ from suction head 12 and efficient penetration of aortic septum 1000. This served to reduce extension forces and improve advancing of needle 22′ into aortic septum 1000. In some embodiments, beveled distal end 23 can define an angle greater than an angle of guide feature 39 such that the distal tip of beveled distal end 23 is spaced apart from guide feature 39, as illustrated.

As can be seen in FIG. 12, central guide feature 39 serves the additional purpose of directing pliable septal tissue such that it is less oblique to the angle of the needle-wire 22/needle 22′. This decreases the force required to pass the needle-wire 22/needle 22′ through the septal tissue.

In some embodiments, suction head 12 possesses a proximal ring-shaped extrusion 42 over which the outer sheath 14 sits or within which the outer sheath 14 is captured and to which it is attached. In some embodiments, this ring is wedge shaped with a thin portion most proximal and a thick portion most distal and angled to direct the needle in towards the center of suction head 12. In some embodiments, the outer sheath 14 is captured within ring-shaped extrusion 42 to minimize any edge features that may cause operational binding of needle-wire 22/needle 22′ during actuation.

In some embodiments, suction head 12 may include one or more radio-opaque markers 15 (FIG. 23A). In some embodiments, radio-opaque markers 15 are imbedded within suction head 12 to indicate device position and orientation on 2-dimensional fluoroscopy or other X-ray based imaging. In some embodiments, radio-opaque markers 15 are constructed of a radio-opaque material, such as tungsten, gold, platinum, or other material. In some embodiments, radio-opaque markers 15 may be composed of two linear markers positioned laterally to the orifice of suction head 12. In some embodiments, radio-opaque markers 15 are oriented parallel to suction aperture 32 to aid the user in aligning suction head 12 with a tissue plane using fluoroscopy prior to activating suction for tissue attachment to suction head 12, although, other orientations and configurations of radiopaque markers may be used.

### Valved Ports

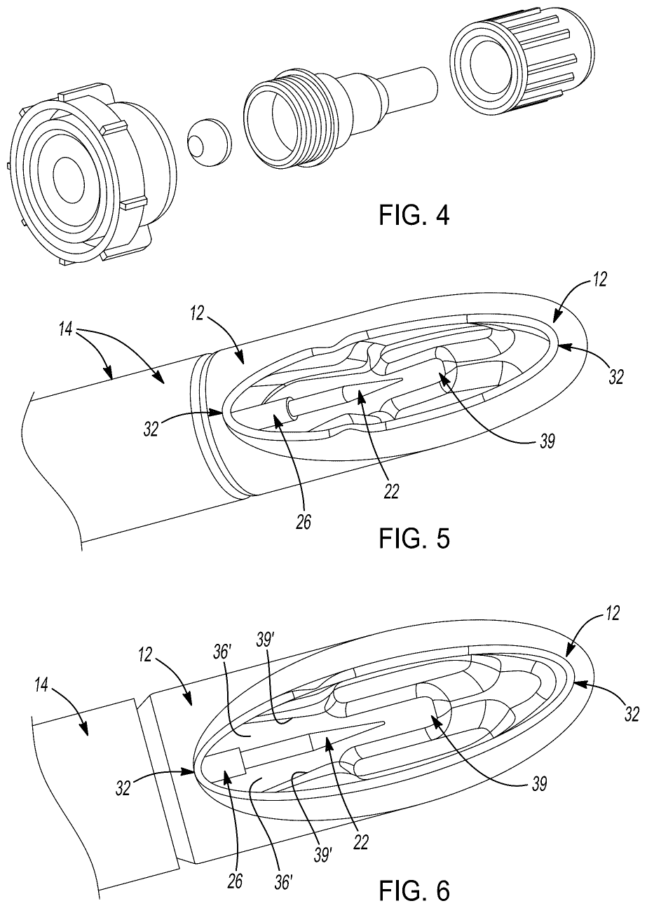

In some embodiments, as illustrated in FIGS. 2 and 4, an external valved port can be located at external/proximal terminus of needle-wire catheter 26. In some embodiments, an additional valve or port can be disposed at proximal terminus of outer sheath 14. Valve port can accept needle-wire catheter 26 and self-seal thereabout to prevent flow of blood in the retrograde direction out of the device and permit smooth passage of needle-wire 22, needle 22′, and needle-wire catheter 26. It should be noted that the valved port may be included as part of an integrated external manifold as discussed herein.

In some embodiments, as illustrated in FIG. 21, a valve port assembly 50 can be located within handle 16 at a proximal terminus of outer sheath 14. In some embodiments, valve port assembly 50 can comprise a distal end 52 bonded or otherwise coupled to a proximal end of outer sheath 14 to form a seal 54 therebetween to maintain a suction within outer sheath lumen 38. As will be discussed herein, valve port assembly 50 is configured to provide a continuous suction tubing or channel between suction source 30, outer sheath lumen 38, and suction head lumen 36.

### Suction Syringe/Suction Pump

In some embodiments, as illustrated in FIG. 2, suction source 30 can be coupled via Luer lock fitting 20 and configured to provide sufficient suction to adhere suction head 12 to septum 1000 and, in some embodiments, permit the user to reattach the suction head 12 multiple times if dislodged or if multiple fenestration locations are desired.

In some embodiments, suction source 30 is configured to generate negative pressure. In some embodiments, Luer lock fitting 20 and/or suction source 30 can comprise a negative pressure gauge. Suction source 30 can be configured to release negative pressure and regenerate multiple times. In some embodiments, suction source 30 can comprise an electronic aspiration pump or syringe, including a locking syringe. In some embodiments, the suction source 30 can detect when the suction head 12 makes a connection with tissue, for example by detecting an increase in vacuum pressure within the catheter, to notify the user that the suction head is successfully attached. In some embodiments, suction may be externally generated and connected to the device via suction tubing (AKA wall suction). In this case, there may be a suction regulator in-line.

In some embodiments, multiple locking syringes can be placed in series or parallel using a manifold as illustrated in FIGS. 18A and 19. In some embodiments, suction tubing is operably coupled to valve port assembly 50 and exits handle 16 and is operably coupled a multi-way stop cock and/or Luer lock fitting 20 for connection of suction source 30. The suction may be delivered, for example, by first generating a vacuum within the suction source 30, and then, when the operator is ready to deliver the suction at the suction head, by opening of the stopcock 20, although other means of suction activation are contemplated, for example, via an activation button, or other.

Still referring to FIG. 19, in some embodiments, suction source 30 comprises a manifold 31 and at least one locking syringe 33. The manifold 31 includes one or more stopcocks 35, preferably at the connection point of each locking syringe 33, to enable the user to select which vacuum source they want to enter sheath lumen 38 and suction head lumen 36. In some embodiments, a locking syringe is a syringe where the plunger can be pulled back to generate a vacuum and the plunger can then be locked in this pulled back state without the user holding the syringe. This enables the user to selectively deploy a multitude of vacuum sources. In some operations, the user may advance the suction head 12 up to a tissue or surface they desire to fenestrate. The user may then proceed to attempt attachment of the suction head 12 to the tissue. They now open the stopcock 35 on one of the plurality of locking syringes 33. However, if the alignment of the suction head 12 wasn't correct and tissue apposition wasn't obtained, rather than resetting the locking syringe to generate more vacuum, the user instead may reposition suction head 12 and open stopcock 35 on the next locking syringe 33, enabling a more rapid re-attempt at tissue apposition with suction head 12. This manifold can allow the user the generate a surplus of vacuum pressure and deploy it when desired to affix the suction head to the desired tissue.

### Needle-Wire

In some embodiments, needle-wire 22 can comprise an elongated wire having beveled distal end or tip 23, which in some embodiments is tapered and/or other wire surface to permit the user to puncture the septum. A distal end portion of needle-wire 22 may include a pre-curvature to facilitate manipulation. The needle-wire 22 can be of sufficient length such that the distal tip is deployable beyond suction head 12, a diameter of about 0.010-0.040″, and a proximal end is manipulable by the user. In some embodiments, needle-wire 22 can be made of a material including but not limited to stainless steel and/or nitinol, and may be solid or hollow. The needle-wire 22 is further configured to traverse needle-wire catheter 26 and the bend in the suction head 12. The needle wire may be configured to accept passage of a guidewire through its central bore which is advantageous so that, after needle fenestration, a guidewire may be passed directly into the false lumen.

### Needle

In some embodiments, needle 22′ can comprise an elongated hollow needle having beveled distal end or tip 23, which in some embodiments is tapered and/or other surface to permit the user to puncture the septum. A distal end portion of needle 22′ may include a pre-curvature to facilitate manipulation. The needle 22′ can be of sufficient length such that the distal tip is deployable beyond suction head 12, a diameter of about 0.010-0.070″, and a proximal end is manipulable by the user. In some embodiments, needle 22′ can be made of a material including but not limited to stainless steel and/or nitinol, and may be hollow. Needle 22′ is further configured to traverse or be advanced through outer sheath 14 and accommodate bending forces. Needle 22′ may be configured to accept passage of guidewire 28 through its lumen such that after needle fenestration, guidewire 28 may be passed directly into the false lumen.

In some embodiments, as illustrated in FIGS. 18A, 19, and 21, a Luer lock fitting 21 disposed at a proximal end of needle 22′ is provided to enable aortic fenestration device 10 to be operated without the presence of guidewire 28 and/or permit guidewire 28 to be introduced after fenestration.

### Needle-Wire Catheter

In some embodiments, needle-wire catheter 26 comprises a thin-walled hollow catheter which slides over needle-wire 22. In some embodiments, needle-wire catheter 26 is of sufficient length such that the needle-wire 22 can be advanced at least 1 cm beyond the suction head 12 with sufficient proximal length for manipulation by the user without buckling. In some embodiments, needle-wire catheter 26 can be made of a low-friction material to facilitate actuation of needle-wire 22.

In some embodiments, needle-wire catheter 26 comprises a proximal portion containing the valved port. In some embodiments, needle-wire catheter 26 is configured to accept standard sized guidewires 28 and permits their advancement into the false lumen, such as but not limited to 0.014″, 0.018″, and/or 0.035″ guidewire 28.

During operation, needle-wire catheter 26 is configured to provide additional stiffness and steerability to needle-wire 22. To this end, needle-wire catheter 26 can slide over needle-wire 22 to cross septum. In some embodiments, needle-wire catheter can include one or more radiopaque markers to help facilitate navigation within the patient. In some embodiments, the needle-wire catheter may include a tapered distal tip.

### Advancement Mechanism

In some embodiments, an advancement mechanism 24 is provided that is configured to extend and/or retract needle-wire 22/needle 22′ within and relative to suction head 12 and, particularly, relative to the septal contacting face or suction aperture 32. In some embodiments, advancement mechanism 24 is configured to actuate and/or position needle-wire 22/needle 22′ into one or more selectively retainable positions. In some embodiments, advancement mechanism 24 maintains an ideal orientation of needle-wire 22/needle 22′ relative the septal wall to minimize penetration force across aortic septum 1000. In some embodiments, advancement mechanism 24 is configured to enable the user to advance needle needle-wire 22/needle 22′ at a discrete distance beyond the suction aperture 32 of suction head 12. In some embodiments, advancement mechanism 24 is configured to enable the user to advance needle-wire catheter 26 to a discrete distance beyond the needle-wire 22. In some embodiments, advancement mechanism 24 is configured to enable the user to retract needle-wire 22 completely through needle-wire catheter 26 so it may be removed. In some embodiments, advancement mechanism 24 is configured to be steerable.

With particular reference to FIGS. 18A-19 and 21, advancement mechanism or actuator device 24 can comprise a slider member 62 operably bonded or otherwise coupled with needle-wire 22/needle 22′ and disposed in sliding arrangement with handle 16 within a slider housing slot 64 to be manually actuated between a retracted position (FIG. 21) and an extended position. In some embodiments, slider member 62 can be optionally locked in place when not depressed using, for example, a ball spring mechanism, a stepped grove mechanism, or others. That is, by pressing down on the slider member 62, slider member 62 is free to translate about the slider member housing slot 64. The slider member advances the needle in 1:1 fashion. The needle-wire 22/needle 22′ is rigidly affixed to the slider member 62. The slider member 62 allows continuous advancement and retraction of needle-wire 22/needle 22′. Then, when the slider member 62 no longer has downward pressure applied, its relative motion to the handle 16 is fixed, preventing needle-wire 22/needle 22′ from linear translation relative to handle 16.

### Guidewire

In some embodiments, guidewire 28 is a soft tip guidewire having a diameter in the range of 0.014″-0.035″, however other sizes may be used. Guidewire 28 may be sized such that it can be linearly translated within needle wire catheter 26 or needle 22′.

### Operation

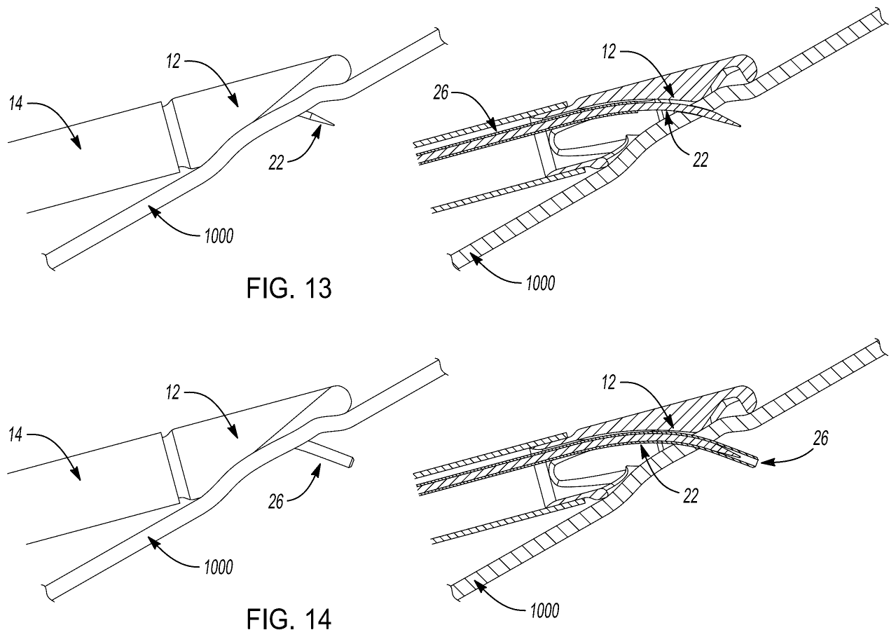

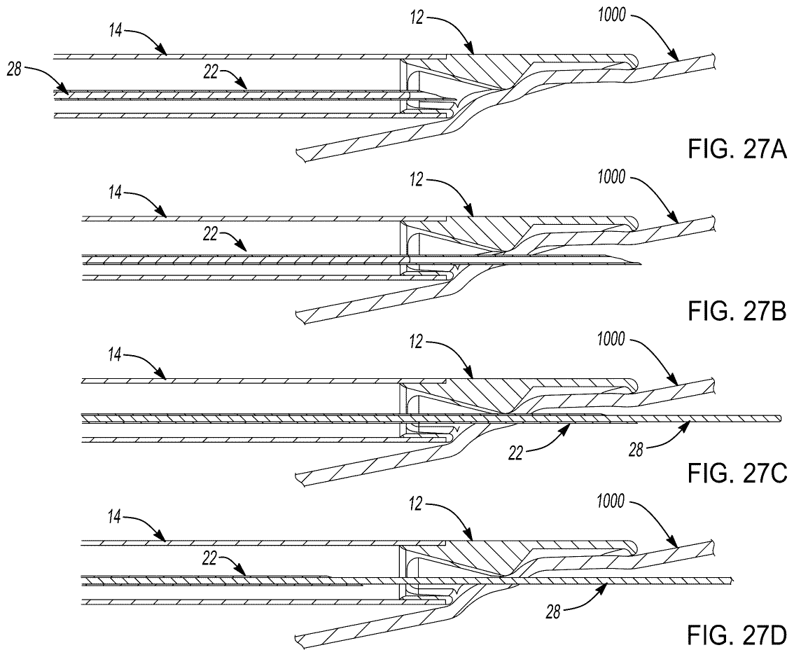

As illustrated in the figures, during operation, such as illustrated in FIG. 11, aortic fenestration device 10 is advanced into abdominal aorta and positioned adjacent to septal wall of aortic septum 1000. As illustrated in FIGS. 12 and 27A, suction is activated and attachment to the aortic septum 1000 is confirmed. It should be noted that the aortic fenestration device 10, or specifically suction head 12, may be attached either within the false lumen or the true lumen depending on the situation. As illustrated in FIGS. 13 and 27B, advancement of needle-wire 22/needle 22′ is guided by guide feature(s) 39, resulting in less oblique puncture angle relative to aortic septum 1000. Needle-wire 22/needle 22′ can be advanced across aortic septum 1000 and, in some embodiments, needle-wire catheter 26 remains housed within suction head 12.

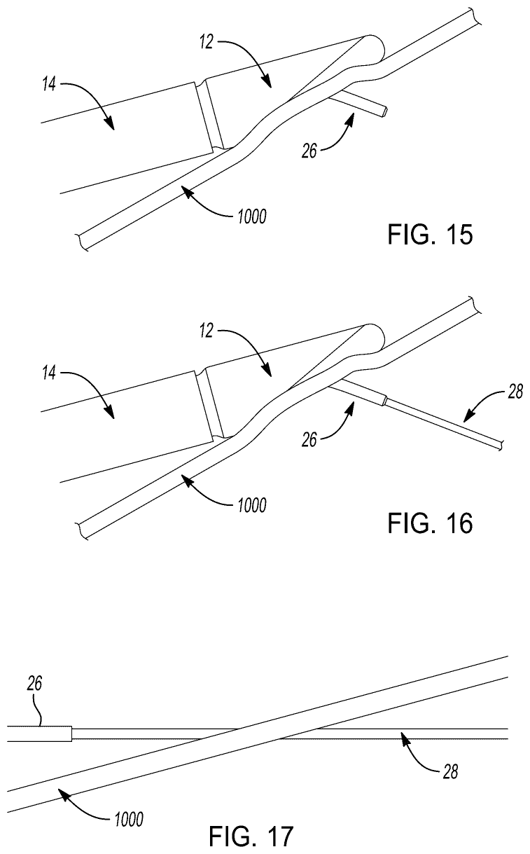

As illustrated in FIG. 14, needle-wire 22 can remain fixed in place and needle-wire catheter 26 can be advanced over the needle-wire 22 and across aortic septum 1000. As illustrated in FIG. 15, needle-wire 22 can then be removed to leave needle-wire catheter 26 in place within the false lumen. Guidewire 28 can then be placed through the needle-wire catheter 26 or needle 22′ and into the second lumen, as illustrated in FIGS. 16 and 27C. As illustrated in FIGS. 17 and 27D, suction can be terminated and the device removed over the guidewire 28, thereby leaving the guidewire 28 in place across the aortic septum 1000 and with its distal extent in the second lumen. Guidewire access to the false lumen permits the operator to proceed with a wide range of maneuvers to treat malperfusion, such as dilation and stenting as well septostomy for optimization of an endovascular graft landing zone in chronic type B dissection.

### Alternative Concepts

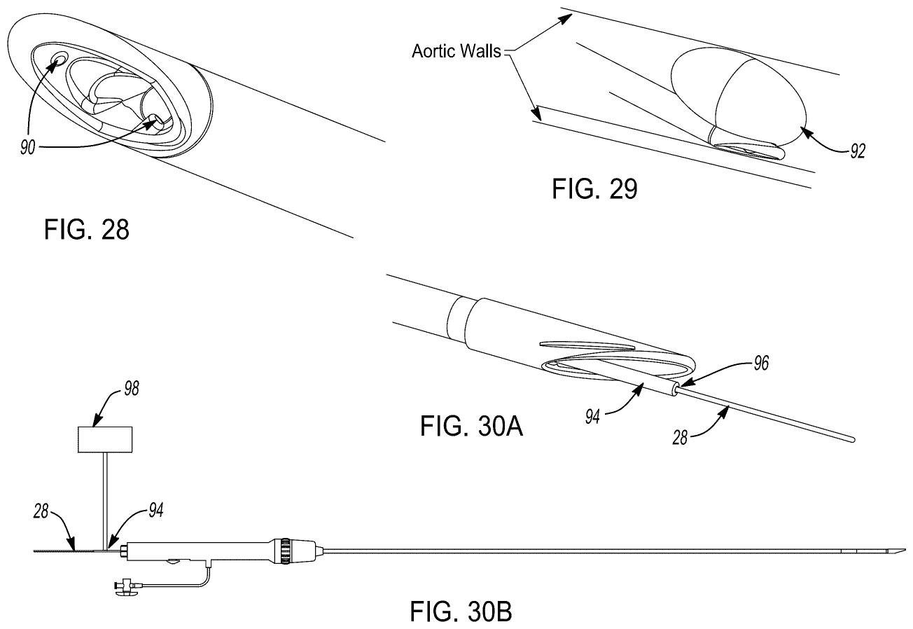

In some embodiments, as illustrated in FIG. 28, suction head 12 can include a pair of bipolar electrodes 90. As an example, when the suction head is affixed to the tissue, energy can be transmitted to the bipolar electrodes. One or more wires can be connected to the electrode(s) and be incorporated in the wall of the outer sheath. The user can then use an external power source to connect to the wires and power the electrodes. When the bipolar function is activated by the user, the electrical current will then flow between the electrodes, cutting the tissue between and enabling the passage of a guidewire across the cut surface. Additional alternative means of cutting or fenestration the tissue engaged at the suction aperture can include unipolar electrodes, a harmonic scalpel which uses high frequency vibrations to cut tissue, or other means.

As illustrated in FIG. 29, alternative means of biasing the device tip to one side of the vessel lumen, such as with an inflatable member 92, like a balloon, which may be inflated at the user's discretion via a secondary lumen within the outer sheath to direct or bias the tip of the device. An alternate means of biasing the tip may include expansile components, precurvature of the outer sheath, heat responsive curvature of the outer sheath, and others.

As described herein, the principles of the present teachings may be used with a laser device 94, as illustrated in FIG. 30, in place of the needle-wire 22/needle 22′. In such embodiments, the needle component is replaced with a catheter-based laser 94, such as a coronary laser atherectomy catheter (such as, for example ECLA laser by Philips). The laser catheter can include an energized tip 96 with an internal lumen capable of accepting guidewire 28. When the laser is activated at the source 98, the catheter-based laser can be advanced through the tissue, similar to that shown in earlier embodiments. A guidewire can then be passed through the inner lumen of the catheter-based laser and into the false lumen or other cavity. The catheter-based laser can be connected to an external power source.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An aortic fenestration device comprising:
an outer sheath;
a suction head being operably coupled to a distal end of the outer sheath, the suction head having a suction aperture;
a fenestration element disposed within the outer sheath, the fenestration element configured to fenestrate tissue;
a guidewire slidably disposed relative to the fenestration element and extendable to a position beyond the suction aperture and a distal end of the fenestration element; and
a suction source operably coupled to the outer sheath and configured to exert suction pressure at the suction aperture of the suction head to selectively couple the suction head to the tissue via suction pressure as the tissue is fenestrated.

2. The aortic fenestration device according to claim 1 wherein the fenestration element comprises a hollow needle.

3. The aortic fenestration device according to claim 2 wherein the hollow needle is slidably disposed within the outer sheath and extendable through the suction head to a position beyond the suction aperture.

4. The aortic fenestration device according to claim 3 wherein the guidewire is slidably disposed within the hollow needle and extendable through the hollow needle to a position beyond the suction aperture and a distal end of the hollow needle.

5. The aortic fenestration device according to claim 1 wherein the fenestration element comprises:
a needle-wire catheter slidably disposed within the outer sheath and extendable through the suction head to a position beyond the suction aperture; and
a needle-wire slidably disposed within the needle-wire catheter and extendable through the needle-wire catheter to a position beyond the suction aperture and a distal end of the needle-wire catheter.

6. The aortic fenestration device according to claim 1 further comprising:
an advancement mechanism configured to extend or retract fenestration element relative to the suction aperture of the suction head.

7. The aortic fenestration device according to claim 6 wherein the advancement mechanism is configured to actuate or position fenestration element into one or more selectively retainable positions.

8. The aortic fenestration device according to claim 1 wherein the outer sheath is configured to articulate about a longitudinal axis thereof.

9. The aortic fenestration device according to claim 8 wherein the outer sheath is articulated in response to contact with a guide feature formed within the suction head.

10. The aortic fenestration device according to claim 8, further comprising an outer sheath articulating assembly configured to actively articulate outer sheath about the longitudinal axis, the outer sheath articulating assembly having a drive member operably actuating one or more wire members disposed along a length of the outer sheath, the outer sheath articulating assembly configured to exert a bending force at at least one zone of the outer sheath in response to movement of the drive member.

11. The aortic fenestration device according to claim 10 wherein the outer sheath articulating assembly is configured to exert a bending force within the outer sheath to produce articulation of the suction head in the direction of the suction aperture.

12. The aortic fenestration device according to claim 1 wherein the suction head comprises an internal suction head lumen fluidly coupled to the suction aperture,
wherein the suction source is configured to exert suction pressure at the suction aperture of the suction head via the internal suction head lumen.

13. The aortic fenestration device according to claim 1 wherein the suction head comprises one or more guide features therein configured to direct the fenestration element toward a center of the suction aperture.

14. The aortic fenestration device according to claim 13 wherein the one or more guide features comprise sloped surfaces.

15. The aortic fenestration device according to claim 13 wherein the one or more guide features comprise a central sloped surface and at least a pair of lateral sloped surfaces.

16. The aortic fenestration device according to claim 15 further comprising a suction head lumen formed between the central sloped surface and at least one of the at least a pair of lateral sloped surfaces.

17. The aortic fenestration device according to claim 15 further comprising a suction head lumen formed as a channel extending through the central sloped surface.

18. The aortic fenestration device according to claim 1, further comprising a valve port assembly operably coupled between the suction source and the outer sheath.

19. The aortic fenestration device according to claim 1 wherein the suction source comprises a manifold fluidly coupled to at least one locking syringe.

20. The aortic fenestration device according to claim 1 wherein the fenestration element is extendable through the suction head to a position beyond the suction aperture.
